(19) United States
(12) Patent Application Publication
(10) Pub. No.: US 2016/0183801 A1
(43) Pub. Date: Jun. 30, 2016

(54) Polarized OCT with Improved SNR

(71) Applicant: Joshua Noel Hogan, Los Altos, CA (US)

(72) Inventor: Joshua Noel Hogan, Los Altos, CA (US)

(21) Appl. No.: 14/975,745

(22) Filed: Dec. 19, 2015

Related U.S. Application Data
(60) Provisional application No. 62/096,909, filed on Dec. 26, 2014.

(51) Int. Cl. A61B 5/00

(52) U.S. Cl. CPC A61B 5/0066

(57) ABSTRACT

The invention provides a polarized optical system with minimal use of wave plates that provides isolation of the optical source and has optimized signal to noise performance. The polarized optical system in the preferred embodiment is an interferometric optical system, and in particular an optical coherence tomography (OCT) system. Various alternate embodiments are provided.

### CROSS REFERENCES TO RELATED PATENTS OR APPLICATIONS

This patent application, docket number 141101US, claims priority from U.S. provisional application No. 62/096,909, docket number CI141101PR filed on 26 Dec 2014; and is related to U.S. Pat. No. 7,526,329 titled Multiple Reference Non-invasive Analysis System; U.S. Pat. No. 7,751,862 titled Frequency Resolved Imaging System; and U.S. Pat. No. 8,310,681 titled Orthogonal reference analysis system with enhanced SNR.

### FIELD OF THE INVENTION

The invention described and illustrated in this application relates to the field of non-invasive imaging and analysis and measurement of targets, such as tissue, to image the tissue or to measure the concentration of analytes. In particular the invention relates to the use of non-invasive technologies, such as, Optical Coherence Tomography (OCT) to image and analyze tissue including, but not limited to, skin tissue and retinal tissue. Such analysis includes using OCT to determine the concentration of analytes such as glucose in tissue or tissue fluids.

### BACKGROUND OF THE INVENTION

OCT is commonly used to image tissue for ophthalmic analysis, such as retinal imaging and analysis. OCT is also used to image skin tissue and OCT has been explored as a technique for measuring glucose concentration. For example U.S. Pat. No. 6,725,073 by Motamedi , et al., titled “Methods for noninvasive analyte sensing” describes using OCT to measure glucose concentration.

A polarized multiple reference system OCT system, consistent with prior art, is depicted in FIG. 1. A broadband optical source, such as a super-luminescent diode (SLD) and lens combination 101 emits a collimated optical beam 102 which is transmitted through an optional polarizer 103 through a half-wave plate 104 and split by a polarized beam-splitter 105 into reference radiation106 and probe radiation 112.

The reference radiation 106 is transmitted through an attenuator 107 and a quarter wave plate 108 and then partially through a partial reflective mirror 109 to a reference mirror 110 mounted on a oscillating translation device 111, such as a voice coil or piezo device. The combination of the partial mirror 109 and the reference mirror 110 generates multiple reference signals as described in the patents incorporated herein by reference.

As the reflected reference radiation is transmitted back through the quarter wave plate 108 its polarization vector is rotated such that it will be re-directed by the polarized beam-splitter 105 towards the detection system depicted in the dashed box of FIG. 1.

The probe radiation 112 is transmitted through a second quarter wave plate 113 and through an anti-reflection coated blank that compensates for effects of the optical elements in the reference path. The probe radiation 112 is scattered by components in the target 115. Some of the probe radiation is scattered back through the quarter wave plate 113 where the double pass through the quarter wave plate 113 rotates its polarization vector by ninety degrees thereby enabling this scattered probe radiation to be transmitted through the polarized beam-splitter 105 towards the detection system.

The combined scattered probe radiation and reflected reference radiation is transmitted through an optional second half wave plate 116 to a second polarized beam splitter 117 that reflects one set of components of the reflected reference and scattered probe radiation to a detector 118 and transmits the orthogonal set of components of the reflected reference and scattered probe radiation to a detector 119 thereby achieving balanced detection.

In some embodiments the optional second half wave plate 116 is not present but the second polarized beam splitter 117 is rotated forty five degrees about the optical beam 120 so that again the polarized beam splitter 117 reflects one set of components of the reflected reference and scattered probe radiation to a detector 118 and transmits the orthogonal set of components of the reflected reference and scattered probe radiation to a detector 119.

Operation of the OCT system is controlled by means of a control module 121. The detected signals are processed by a processing module 122 to yield imaging and analysis of the target.

This approach requires two quarter wave plates in order to rotate the reflected reference and back-scattered probe radiation so that the beams that form the OCT signal are directed towards the detection system by the polarized beam-splitter 105. Polarized OCT systems have an advantage over non-polarized OCT systems in that they can readily yield the orthogonal or complimentary signals that are required for balanced detection.

Furthermore since typical polarized OCT systems direct substantially all of the reflected reference and back-scattered probe radiation towards the detection system, they thereby isolate the optical source from undesirable optical feed-back. While this reduces source noise, it contributes to detector noise. In the conventional MRO system, the reference signal from the lower orders have greater magnitude which is not optimal for all targets. Furthermore, light scattered from the front surface of a target, such as tissue, generates additional detector noise. What is needed is a means for reducing detector and source noise, as well as a means of enhancing signals.

Moreover, in conventional systems, quarter wave plates are used to maximize light to the detector, both reference and back scattered light from the target. Quarter wave plates also direct noise generating light to the detector. Moreover, quarter wave plates are costly components in a system, particularly in the case of OCT systems where broadband OCT quarter wave plates are required. There is therefore an unmet need for a polarized OCT system with fewer or no quarter wave plates but which has the optical source isolated from undesirable optical feed-back and maintains an optimal signal to noise ratio.

### SUMMARY OF THE INVENTION

The invention described herein provides a polarized optical coherence tomography (OCT) system with minimal use of wave plates. The system optimizes the signal to noise characteristics of the OCT signals and provides isolation of the optical source from undesirable optical feed-back. The probe path does not have a quarter wave plate and transmits, to the detection system, only the polarization component of the scattered probe radiation that is orthogonal to the original probe radiation. In one embodiment the reference path does not have a quarter wave plate and uses a reference mirror with a thin wave plate to rotate components of the reference radiation to yield components of the reference radiation that are orthogonal to the original reference radiation and therefore are directed towards the detection system. A polarizer in the source path and an optional attenuator in the reference path reduce feedback to the optical source. In some embodiments a pilot signal is detected to assist with system alignment and signal and data processing.

The invention provides an improved optical coherence tomography system. In an OCT system including a radiation source; a radiation detector; in the pathway of the radiation between the source and the detector, a means for directing radiation along a reference path and along a probe path and wherein at least one quarter wave plate is positioned in the reference path, and a means to capture returning probe and reference radiation and to process interferometric signals therefrom, the improvements are three fold a) using a coated reference mirror, where the coating functions as a thin wave plate, and replaces the quarter wave plate in said reference path, and thereby reducing detector noise; b) a polarizer orientated relative to the beam splitter 205 such that a small percentage of the source radiation is directed along the reference path and substantially all the radiation is directed toward the target, and such that the radiation reflected from the partial mirror 209 is substantially attenuated by the polarizer, hence isolating the source from reflected reference radiation and thereby reducing source noise; and c) a probe path from the beamsplitter 205 to the target 215 such that polarization remains unchanged, such that only radiation backscattered from the target whose polarization is rotated has a component that passes through beamsplitter 205 to the detector, thereby reducing detector noise attributable to the target.

### BRIEF DESCRIPTION OF THE DRAWINGS

The drawing intended as an aid to understanding the invention:

FIG. 1 is an illustration of prior art depicting a polarized OCT system using a conventional quarter wave plate based design that isolates the optical source and directs reflected reference radiation and back-scattered probe radiation towards the (balanced) detection system.

FIG. 2 is an illustration of an embodiment of the invention depicting an OCT system that uses a coated reference mirror 210, where the coating functions as a thin wave plate, to direct the useful reference radiation towards the detection system. The polarized beam splitter 205 depicted in this embodiment directs radiation back-scattered from the target, where the radiation has a polarization vector orthogonal to the polarization vector of the probe radiation applied to the target. The inventive system includes polarizer 203 which isolates the optical source from undesirable optical feedback. This embodiment also provides for detection of a pilot signal to provide real time feed-back for aligning the system and improved processing of signals and data.

FIG. 3 is an illustration of both an embodiment with an alternate scheme for detection of a pilot signal to provide real time feed-back for aligning the system and improved processing of signals and data.

### DESCRIPTION OF A PREFERRED EMBODIMENT OF THE INVENTION

The invention described herein provides a method, apparatus and system for a polarized optical system with minimal use of wave plates that provides isolation of the optical source and has optimized signal to noise performance. The polarized optical system is typically an interferometric optical system such as an optical coherence tomography (OCT) system.

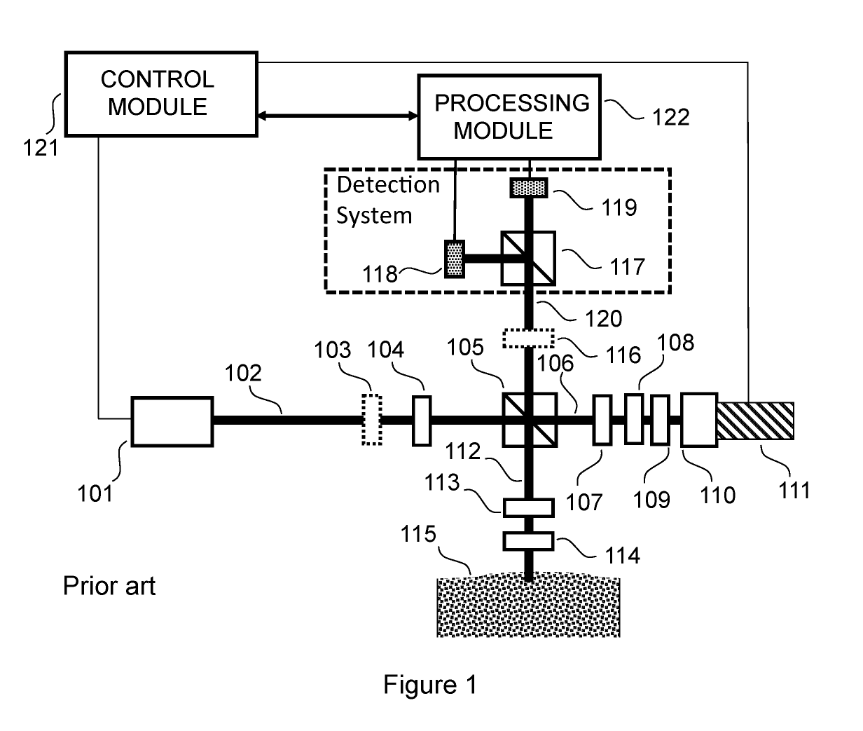

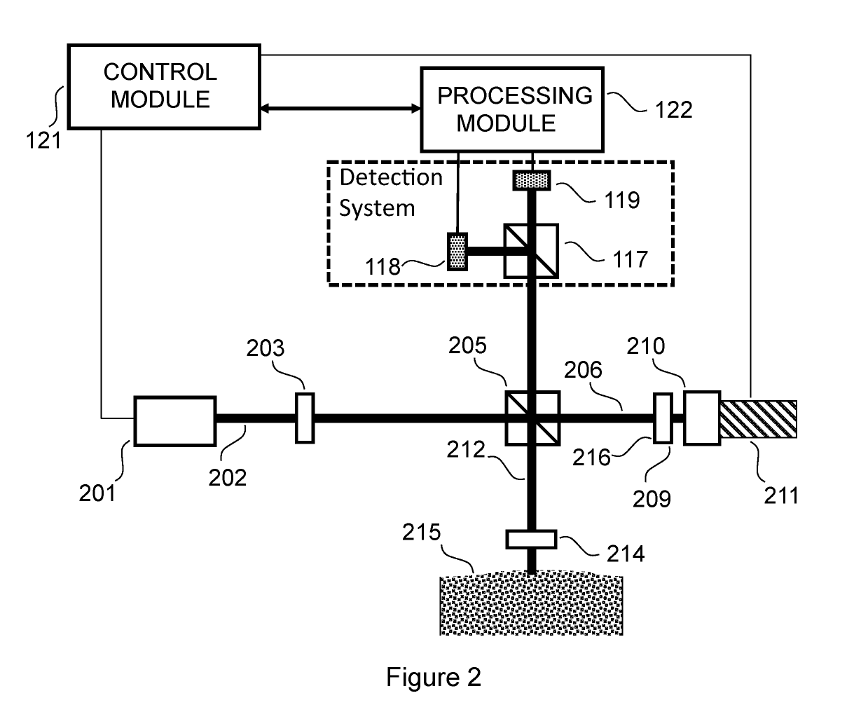

A preferred embodiment consists of a polarized multiple reference system OCT system, and is depicted in FIG. 2 where a broadband optical source, such as a super-luminescent diode (SLD) and lens combination 201 emits a collimated optical beam 202 which is transmitted through a polarizer 203 to a polarized beam-splitter 205 which splits the optical beam into (original) reference radiation 206 and (original) probe radiation 212. Note some aspects depicted in FIG. 2 that are the same as in FIG. 1 are labeled with the same numbers as in FIG. 1.

The angular orientation of the polarizer 203 about the collimated optical beam 202 determines the ratio of the optical power split of the reference radiation 206 and probe radiation 212. Typically reference radiation 206 has substantially lower power than the probe radiation 212 (less than 0.1%). The angular orientation of the SLD about the line of the collimated optical beam 202 is set to maximize the amount of radiation transmitted through the polarizer 203.

The reference radiation 206 is partially transmitted through a partial reflective mirror 209 to a reference mirror 210 mounted on a oscillating translation device 211, such as a voice coil or piezo device. The combination of the partial mirror 209 and the reference mirror 210 generates multiple reference signals as described in the patents incorporated herein by reference.

A substantial portion (typically in the range of 80% to 95%) of the original reference radiation 206 that is initially reflected by the partial mirror 209 is transmitted back through the beam-splitter 205 to the polarizer 203, since the polarization vector of this reflected radiation is substantially unchanged.

The angular orientation of the polarizer 203 will transmit less than 0.1% of this portion of the reference radiation (less than 0.0001% of the original collimated beam 202 output by the optical source) thereby effectively isolating the optical source from this portion of the reference radiation.

Furthermore, since substantially all of this portion of the reference radiation is directed back towards the source and not redirected along the detection path, it does not contribute to detector noise.

In the preferred embodiment the reference mirror 210 mounted on a oscillating translation device 211 has a coating designed to rotate the plane of polarization of incident radiation. In the preferred embodiment, the coating is a thin wave plate that rotates the plane of polarization of incident radiation by a small angle (less than five degrees). The combination of the partial mirror 209 and the reference mirror 210 generates multiple reference signals as described in the patents incorporated herein by reference.

The rotation due to the thin wave plate causes successive reflections of the reference radiation (between the reference mirror 210 and the partial mirror 209) to have an increasing magnitude of rotation of their respective polarization vectors. This rotation of polarization vectors of successive reflections causes at least some of the reference radiation components transmitted back through the partial mirror to have a polarization component orthogonal to the polarization vector of the original reference radiation 206.

The polarization component parallel to the polarization vector of the original reference radiation 206 is transmitted through the beam-splitter 205. The polarizer 203 acts as an attenuator to also isolate the optical source from this component of the reference radiation.

The (original) probe radiation 212 is transmitted through an anti-reflection coated blank 214 to a target 215 where at least some of the probe radiation is scattered back substantially along the line of the incident probe radiation. Typical embodiments include one or more focusing lenses (not depicted in the Figures) to focus the probe radiation into the target and optionally the reference radiation and also focus radiation onto the detectors.

In the preferred embodiment, where the target 215 is tissue, there is no quarter wave plate in the probe path. Therefore only probe radiation that is scattered back by the target with at least some rotation of the polarization vector will have a polarization component orthogonal to the incident probe radiation 212 and thereby be transmitted through the beam-splitter 205 and available to the detection system.

The probe radiation that is scattered back by the target with a polarization component parallel to the incident probe radiation 212 is directed by the beam-splitter 205 to the polarizer 203. Typically this radiation has a substantial component that will be aligned with the polarizer, however, since this radiation is diffuse back-scattered radiation from (weakly scattering) tissue, it has very low intensity and is not well collimated and therefore has low probability of reaching the optical source. The optical source is therefore effectively isolated from this radiation.

As described above, in the preferred embodiment the un-useful radiation of the reference path is directed away from the detection path back towards the optical path, but is effectively isolated from the source. This reduces the noise contribution of the un-useful reference radiation and provides the opportunity to maximize the signal to noise ratio.

The magnitude of the radiation scattered from the surface of the tissue target and from the region within a depth of less than 100 microns from the surface is significantly larger than the magnitude of radiation scattered from deeper regions. This surface and near surface scattered radiation also has substantially the same polarization as the incident probe radiation and, in the preferred embodiment, is therefore directed away from the detection path back, thereby minimizing its optical noise contribution, and towards the optical path, but is effectively isolated from the source, thereby minimizing undesirable optical feedback effects.

Radiation scattered from deeper within the tissue is more depolarized and therefore these weaker signals have typically random polarization with respect to the polarization of the incident probe radiation. Because the radiation scattered from deeper within the tissue is effectively de-polarized, half of this radiation is comprised of a component that has a polarization vector that is orthogonal to the polarization vector of the incident probe radiation.

Therefore in the preferred embodiment half of the radiation scattered from deeper regions will be transmitted through the bean-splitter 205 to the detection system while a substantial portion of the radiation scattered from the surface and near the surface is substantially directed by the beam-splitter 205 away from the detection system towards the optical source (but effectively isolated from the optical source because it is weak diffusively scattered radiation).

This reduces the noise contribution of the strong surface and near surface radiation and provides the opportunity to maximize the signal to noise ratio of the signals associated with deeper regions within the tissue target.

Other aspects that determine the signal to noise ratio of the preferred embodiment include: the total magnitude of the source optical power transmitted through the polarizer 203; the angular orientation of the polarizer 203 and thereby the ratio of the optical power split of the reference radiation 206 and probe radiation 212; and the magnitudes of the angles of polarization rotation of the reference radiation components and thereby the magnitude of the reference radiation components available for the detection system; and the scattering properties of the target.

The scattering properties of the target are determined by the selection of the target, such as tissue. The magnitudes of the angles of polarization rotation of the reference radiation components are determined by the design of the wave plate coating on the reference mirror. The thickness of the wave plate coating can be selected to rotate the polarization vector of a particular order reference to be rotated by substantially 90 degrees and thereby maximize the component of this order reference that is directed towards the detection system, i.e. the detection component.

For example, if the thickness of the wave plate coating is selected to maximize the detection component of the fourteenth order reference signal, then the magnitude of the lower order reference signals will be minimal for the first, second and third orders. This, in conjunction with the use only of scattered orthogonally polarized signals from the target, enables having reduced magnitude interference signals from the surface and near surface of the target and maximizing the signal to noise ratio of interference signals from the deeper regions of the target.

Interference signals of orders greater than fourteen (in this example) would rapidly decrease since both their reference signals are decreasing in magnitude (due to the wave plate based polarization rotation) and the fact that the intensity of the radiation scattered from the deeper regions decreases with depth.

In some versions of the preferred embodiment interference signals between (a) the reflection from the front surface 216 of the optical element that has the partial mirror 209 and (b) very high order reference signals (such as those centered in the region of the twenty eight order reference signals in this example) are also detected and used to optimize either signal processing or as feedback to align the optical system, or to optimize both.

These interference signals are referred to herein as a “pilot signal”. Detection and processing of the pilot signal enables real time monitoring of the system and enables modifying processing to account for variations in system parameters, such as partial mirror to reference mirror distance, or the motion of the voice-coil. Alternatively, or in addition to, it enables feed-back to control such parameters.

The thickness (in conjunction with the refractive index) of the optical element that has the partial mirror 209 and the front surface 216 can be selected to center the pilot signal on a particular high order reference signals. In the example where the fourteenth reference signal is maximized, an appropriate high order reference signal on which to center the pilot signal would be one greater than the twenty eighth reference signal as it would have a polarization vector component directed to the detection system and the frequency content of the pilot signal interference signals will be substantially higher than those used to image or analyze the target.

In the preferred embodiment, radiation from the front surface 216 relies on “leakage” (or non-perfect extinction ratio) of the beam-splitter 205 to be directed towards the detection system. The magnitude of the radiation from this surface 216 can be (in part) determined by the reflectivity of this surface which, in the preferred embodiment, is an un-coated surface, but in other embodiments can have a specified reflectivity including that of an anti-reflection coating.

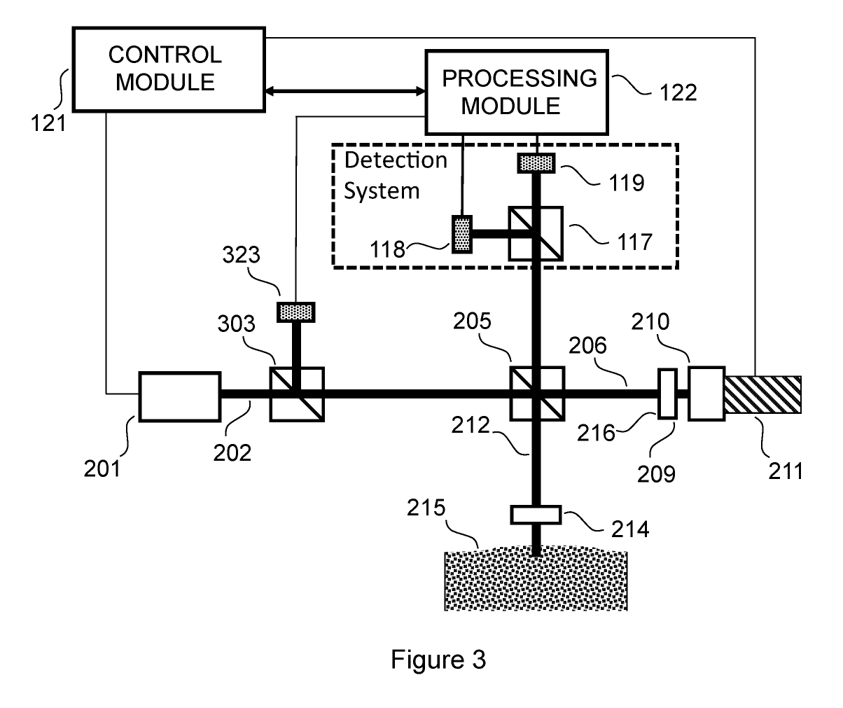

An alternate embodiment is depicted in FIG. 3, which in many respects (indicated by use of the same numbers) is the same as FIG. 2, however the polarizer 203 of FIG. 2 is replaced by the polarized beam-splitter 303 in FIG. 3. The beam-splitter 303 is rotated about the optical beam 202 to determine the ratio of reference radiation to probe radiation and plays a similar role as the polarizer 202 in isolating the optical source.

In this embodiment the reflection from the surface 216 which is substantially transmitted back through the beam-splitter 205 and a significant portion is redirected by the polarized beam-splitter 303 to the detector 323 where it is detected in conjunction with high order reference signals by the a detector 323 to form a pilot signal.

In this embodiment an appropriate high order reference signal on which to center the pilot signal would be the twenty eighth reference signal as it would have a strong polarization vector component transmitted through the beam-splitter 205 and directed to the detector 323 system by the beam-splitter 303. Furthermore the frequency content of the pilot signal interference signals will also be substantially higher than those being use to image or analyze the target.

Other embodiments suitable for other targets can use a reference mirror different from that containing a thin wave plate. For example, in an application that images or analyses retinal tissue the deeper retinal pigment epithelium (RPE) layer has stronger scattering than the inner limiting membrane (ILM) at the surface of the retina.

An embodiment optimized for this application uses, instead of a thin wave plate, a reference mirror 210 that includes a polarizing element aligned at substantially forty five degrees with respect to the orientation of the polarization vector of the (original) reference radiation 206. This causes fifty percent of the reflected reference radiation that reaches the beam-splitter 205 to be directed towards the detection system.

In this embodiment, the systematically diminishing magnitude of the reference radiation with higher order is more optimally suited to the higher scattering (or reflectivity) of the deeper RPE layer of the target. This embodiment could also include an attenuator in the reference path between the beam-splitter 205 and the optical element with the partial mirror 209.

More generally in these embodiments the OCT system includes a wave plate or polarizer to direct the optimized amounts of reference radiation towards the detection system. Other embodiments could have a conventional reference mirror surface 210 and have a conventional quarter wave plate between the beam-splitter 205 and the optical element with the partial mirror 209 or a conventional isolator such as a Faraday isolator.

The above embodiments include a multiple reference OCT system which has un-useful reference radiation reflected from the partial mirror (at the zero order reflection). Polarized versions of other OCT systems including, but not limited to, conventional time domain OCT, Fourier domain swept source OCT, Fourier domain spectrometer based OCT (spectral domain), can avail of some aspects of the invention, such as isolation of the optical source and use of the back scattered radiation from the target where such radiation has a polarization vector orthogonal to the probe radiation applied to the target.

While the invention has been described by means of specific embodiments and applications thereof, it is understood that numerous modifications and variations could be made thereto by those skilled in the art without departing from the spirit and scope of the invention. It is therefore to be understood that within the scope of the claims, the invention may be practiced otherwise than as specifically described herein.

We claim:

1. An improved optical coherence tomography system said system including a radiation source, a radiation detector, in the pathway of the radiation between the source and the detector, a means for directing radiation along a reference path and along a probe path and wherein at least one quarter wave plate is positioned in the reference path, and a means to capture returning probe and reference radiation and to process interferometric signals therefrom, said improvement comprising:
a coated reference mirror, said coating functioning as a thin wave plate, and replacing said quarter wave plate in said reference path, and thereby reducing detector noise;
a polarizer, said polarizer orientated relative to the beam splitter 205 such that a small percentage of the source radiation is directed along the reference path and substantially all the radiation is directed toward the target, and such that the radiation reflected from the partial mirror 209 is substantially attenuated by said polarizer, hence isolating the source from reflected reference radiation and thereby reducing source noise; and
a probe path from the beamsplitter 205 to the target 215 such that polarization remains unchanged, such that only radiation backscattered from the target whose polarization is rotated has a component that passes through beamsplitter 205 to the detector, thereby reducing detector noise attributable to the target.
